(19) United States
(12) Patent Application Publication
(10) Pub. No.: US 2018/0028159 A1
(43) Pub. Date: Feb. 1, 2018

(54) REARWARD ACOUSTIC DIFFUSION FOR ULTRASOUND-ON-A-CHIP TRANSDUCER ARRAY

(71) Applicant: Butterfly Network, Inc., Guilford, CT (US)

(72) Inventors: Matthew R. Hageman, Hoffman Estates, IL (US); Christopher Thomas McNulty, Guilford, CT (US)

(73) Assignee: Butterfly Network, Inc., Guilford, CT (US)

(21) Appl. No.: 15/223,550

(22) Filed: Jul. 29, 2016

(51) Int. Cl. A61B 8/00; H04R 23/00; A61N 7/02

(52) U.S. Cl. CPC A61B 8/546; A61B 8/4483; A61N 7/02; H04R 23/00

(57) ABSTRACT

A heat sink device has a non-planar mounting surface and an ultrasonic transducer substrate attached to the non-planar mounting surface. The non-planar mounting surface of the heat sink device is configured to diffuse acoustic waves that are incident thereupon.

### BACKGROUND

The present disclosure relates generally to ultrasound technology. In particular, the present disclosure relates to an apparatus and method for rearward acoustic diffusion for an ultrasound-on-chip transducer array.

Ultrasound devices may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using a probe), sound waves are reflected off the tissue with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

In one embodiment, a heat sink device has a non-planar mounting surface and an ultrasound-on-chip device attached to the non-planar mounting surface, the ultrasound-on-chip device including an ultrasonic transducer substrate bonded to an integrated circuit substrate. The non-planar mounting surface of the heat sink device is configured to diffuse acoustic waves that are incident thereupon.

In another embodiment, an ultrasound probe includes a housing and an ultrasonic transducer assembly disposed within the housing, the ultrasonic transducer assembly further including a metal heat sink device having a non-planar mounting surface, and an ultrasonic transducer substrate attached to the non-planar mounting surface of the heat sink device. The non-planar mounting surface of the heat sink device may be configured to diffuse acoustic waves that are incident thereupon.

### BRIEF DESCRIPTION OF THE DRAWINGS

Various aspects and embodiments of the disclosed technology will be described with reference to the following Figures. It should be appreciated that the figures are not necessarily drawn to scale. Items appearing in multiple figures are indicated by the same reference number in all the figures in which they appear, and where:

FIG. 1 is a perspective view of a handheld ultrasound probe suitable for use with exemplary embodiments;

FIG. 2 is an exploded perspective view of the ultrasound probe of FIG. 1;

FIG. 3 is a partial cross-sectional view of an exemplary ultrasound-on-chip device suitable for use with exemplary embodiments;

FIG. 4 is perspective view of the ultrasonic transducer assembly shown in FIG. 2;

FIG. 5 illustrates the ultrasonic transducer assembly shown in FIG. 2, with the acoustic lens removed; and

FIG. 6 illustrates the ultrasonic transducer assembly shown in FIG. 5, with the ultrasound-on-chip device removed to reveal a heat sink having a planar mounting surface;

FIG. 7 illustrates a heat sink design in accordance with an exemplary embodiment, including a non-planar mounting surface;

FIG. 8 is an enlarged view of the heat sink design shown in FIG. 7;

FIG. 9 is a top view illustrating the pattern of the non-planar mounting surface;

FIG. 10 is a schematic cross-sectional view illustrating transmission of acoustic energy by an ultrasonic transducer assembly, according to embodiments, in forward and rearward directions;

FIG. 11 is an enlarged view of an ultrasound-on-chip/heat sink interface in FIG. 10;

FIG. 12 illustrates an alternative embodiment of the pattern of the non-planar mounting surface;

FIG. 13 illustrates another alternative embodiment of the pattern of the non-planar mounting surface;

FIG. 14 is a perspective view of another type of ultrasound probe suitable for use with exemplary embodiments;

FIG. 15 illustrates the ultrasound probe of FIG. 14 affixed to a patient;

FIG. 16 is a top view illustrating an alternative fastening mechanism for the ultrasound probe of FIG. 14;

FIG. 17 illustrates the ultrasound probe of FIG. 14 affixed to the patient; and

FIG. 18 is an exploded perspective view of the ultrasound probe of FIG. 16.

### DETAILED DESCRIPTION

Medical ultrasound imaging transducers are used to transmit acoustic pulses that are coupled into a patient through one or more acoustic matching layers. After sending each pulse, the transducers then detect incoming body echoes. The echoes are produced by acoustic impedance mismatches of different tissues (or tissue types) within the patient which enable both partial transmission and partial reflection of the acoustic energy. Exemplary types of ultrasonic transducers include those formed from piezoelectric materials or, more recently, capacitive micromachined ultrasonic transducers (CMUTs) that are formed using a semiconductor substrate.

In the case of a CMUT device, a flexible membrane is suspended above a conductive electrode by a small gap. When a voltage is applied between the membrane and the electrode, Coulombic forces attract the flexible membrane to the electrode. As the applied voltage varies over time, so does the membrane position, thereby generating acoustic energy that radiates from the face of the transducer as the membrane moves. However, in addition to transmitting acoustic energy in a forward direction toward the body being imaged, the transducers simultaneously transmit acoustic energy in a backward direction away from the patient being imaged. That is, some portion of the acoustic energy is also propagated back through the CMUT support structure(s), such as a silicon wafer for example.

When an incident ultrasound pulse encounters a large, smooth interface of two body tissues with different acoustic impedances, the sound energy is reflected back to the transducer. This type of reflection is called specular reflection, and the echo intensity generated is proportional to the acoustic impedance gradient between the two mediums. The same holds true for structures located in a direction away from the patient being imaged, such as a semiconductor chip/metal heat sink interface. If an incident ultrasound beam reaches an acoustic interface at substantially a normal angle (90°), almost all of the generated echo will travel back toward the originating transducer.

Typically, for both piezoelectric and capacitive transducer devices, an acoustic backing material is positioned on a back side of an ultrasonic transducer array in order to absorb and/or scatter as much of the backward transmitted acoustic energy as possible and prevent such energy from being reflected by any support structure(s) back toward the transducers and reducing the quality of the acoustic image signals obtained from the patient by creating interference. In general, however, materials that have good acoustic attenuating and scattering properties may also have poor thermal conductivity and/or coefficient of thermal expansion (CTE) mismatches with respect to the transducer substrate material. Accordingly, exemplary embodiments disclosed herein introduce a heat sink device on which an ultrasonic transducer may be attached that provides both acoustic attenuation/scattering capability, as well as thermal dissipation capability. In one embodiment, a metal heat sink device (e.g., copper) may have a non-planar mounting surface and an ultrasound-on-chip substrate attached to the non-planar mounting surface of the heat sink device.

As compared to a planar surface, the non-planar mounting surface of the heat sink device can be configured to reduce the amount of acoustic energy reflected off the face of the heat sink device and directed back through the body of the semiconductor substrate toward the transducers. Where the angle of incidence with a specular boundary is less than 90°, the echo will not return to the originating transducer; rather, it is reflected at an angle equal to the angle of incidence (similar to visible light reflecting in a mirror). Moreover, in contrast to an acoustic backing that physically separates the transducer substrate from the heat sink surface, a portion of the exemplary heat sink surface may have direct physical contact with the chip surface. Although heat sinking performance may be optimized using a planar surface with maximum surface area contact between the heat sink and the substrate, this comes at a cost of acoustic performance. Therefore, with such a tradeoff, both the benefits of rearward acoustic diffusion and heat dissipation may be achieved.

Embodiments of the present disclosure are described below with reference to the accompanying drawings, in which some, but not all, embodiments of the present disclosure are shown. The present disclosure can be embodied in many different forms and should not be construed as limited to the embodiments set forth herein. Rather, these embodiments are provided so that this disclosure clearly satisfies applicable legal requirements. Like numbers refer to like elements throughout.

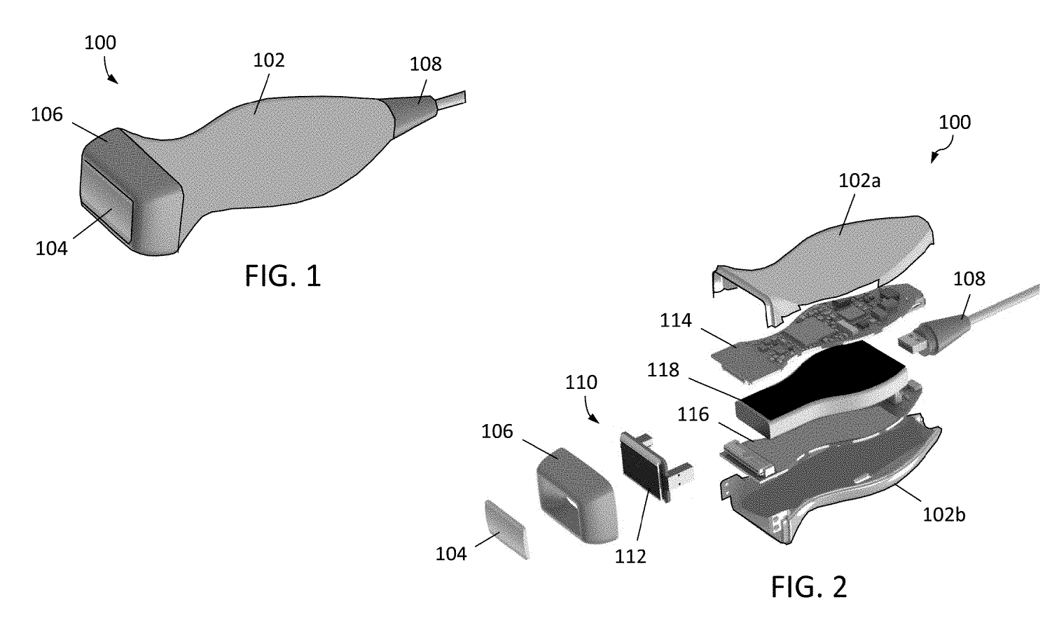

Referring initially to FIG. 1 and FIG. 2, an exemplary ultrasound probe 100 is depicted in a perspective view and an exploded perspective view, respectively. It should be understood, however, that the ultrasound probe 100 depicted in FIG. 1 and FIG. 2 represents one exemplary application for the acoustic attenuation features described herein, and other form factors, applications and devices are also contemplated. As shown in FIG. 1, the exemplary ultrasound probe 100 is a handheld probe that includes a probe housing 102 having an acoustic lens 104 and shroud 106 disposed at a first end thereof, and a cable assembly 108 disposed at a second end thereof. The shroud 106 prevents direct contact between an ultrasonic transducer assembly 110 (FIG. 2) and a patient (not shown) when the ultrasound probe 100 is used to image the patient.

In addition to imaging, the acoustic lens 104 may also be configured to focus acoustic energy to spots having areas of the size required for high-intensity focused ultrasound (HIFU) procedures. Furthermore, the acoustic lens 104 may acoustically couple the ultrasonic transducer assembly 110 to the patient (not shown) to minimize acoustic reflections and attenuation. In some embodiments, the acoustic lens 104 may be fabricated with materials providing impedance matching between ultrasonic transducer assembly 110 and the patient. In still other embodiments, the acoustic lens 104 may provide electric insulation and may include shielding to prevent electromagnetic interference (EMI). Additionally, the shroud 106 and acoustic lens 104 may provide a protective interface to absorb or reject stress between the ultrasonic transducer assembly 110 and the acoustic lens 104.

As also shown in FIG. 2, the ultrasonic transducer assembly 110 includes an ultrasound-on-chip device 112 having an ultrasonic transducer array that is covered by the acoustic lens 104 when the ultrasound probe 100 is assembled. An interior region of the ultrasound probe 100, encapsulated by upper probe housing section 102a and lower probe housing section 102b, may also include components such as a first circuit board 114, a second circuit board 116 and a battery 118. The circuit boards 114 and 116 may include circuitry configured to operate the ultrasonic transducer arrangement 110 in a transmit mode to transmit ultrasound signals, or receive mode, to convert received ultrasound signals into electrical signals. Additionally, such circuitry may provide power to the ultrasonic transducer assembly 110, generate drive signals for the ultrasonic transducer assembly 110, process electrical signals produced by the ultrasonic transducer assembly 110, or perform any combination of such functions. The cable assembly 108 may carry any suitable analog and/or digital signals to and from circuit boards 114 and 116.

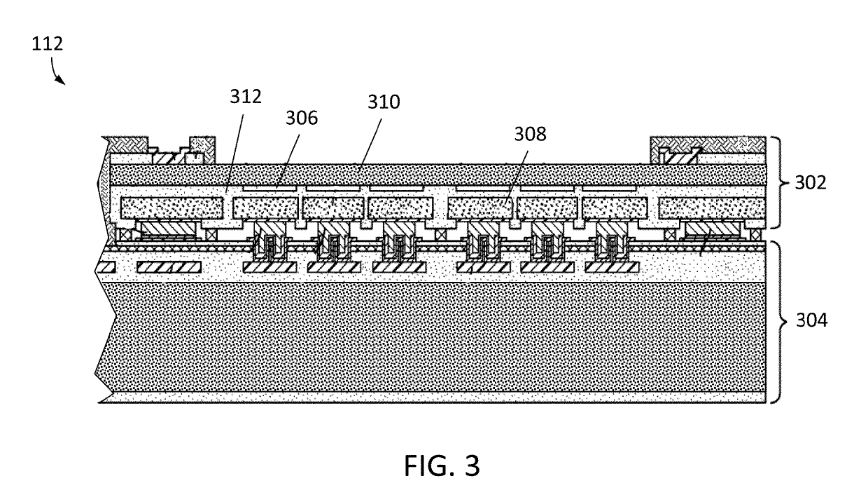

An exemplary configuration for the ultrasound-on-chip device 112 is illustrated in the partial cross-sectional view of FIG. 3. In the embodiment depicted, the ultrasound-on-chip device 112 includes an ultrasonic transducer substrate 302 bonded to an integrated circuit substrate 304, such as a complementary metal oxide semiconductor (CMOS) substrate. The ultrasonic transducer substrate 302 may have a plurality of cavities 306 formed therein, and is an example of a CMUT device as described above. The cavities 306 are formed between a first silicon device layer 308 and a second silicon device layer 310. A silicon oxide layer 312 (e.g., a thermal silicon oxide such as a silicon oxide formed by thermal oxidation of silicon) may be formed between the first and second silicon device layers 308 and 310, with the cavities 306 being formed therein. In this non-limiting example, the first silicon device layer 308 may be configured as a bottom electrode and the second silicon device layer 310 may be configured as a membrane. Thus, the combination of the first silicon device layer 308, second silicon device layer 310, and cavities 306 may form an ultrasonic transducer (e.g., a CMUT), of which six are illustrated in this non-limiting cross-sectional view. To facilitate operation as a bottom electrode or membrane, one or both of the first silicon device layer 308 and second silicon device layer 310 may be doped to act as conductors, and in some cases are highly doped (e.g., having a doping concentration greater than 10¹⁵ dopants/cm³ or greater).

In terms of the aforementioned forward direction toward a subject being imaged, this would be in the upward direction with respect to the view in FIG. 3, whereas the backward direction away from the subject being imaged would be in the downward direction with respect to the view in FIG. 3. Additional information regarding the fabrication and integration of CMUTs with CMOS wafers may be found, for example, in U.S. Pat. No. 9,067,779, assigned to the assignee of the present application, the contents of which are incorporated by reference herein in their entirety. Again however, it should be appreciated that the ultrasonic transducer substrate 302/CMOS substrate 304 embodiment represents just one possible configuration for the ultrasound-on-chip device 112. Other configurations are also possible including, but not limited to, a side-by-side arrangement where transducers and CMOS circuitry are formed on a same substrate, as well as arrays formed from piezoelectric micromachined ultrasonic transducers (PMUTs), or other suitable types of ultrasonic transducers. In still other embodiments, the ultrasound-on-chip device 112 may include an ultrasonic transducer array by itself (i.e., an ultrasonic transducer chip), where CMOS circuitry is located on a different substrate or circuit board altogether.

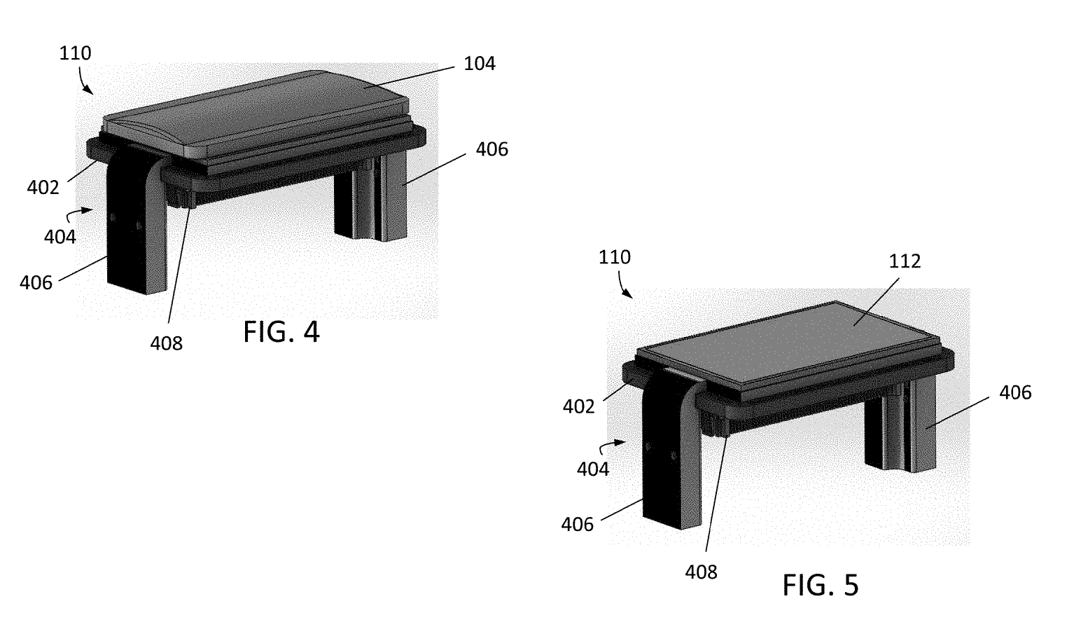

Referring now to FIG. 4, a perspective view of the ultrasonic transducer assembly 110 is illustrated in further detail. In the embodiment shown, the ultrasonic transducer assembly 110 includes an interposer circuit board 402 and a heat sink 404 formed from a thermally conductive material such as copper, for example. In the particular view of FIG. 4, only side tabs 406 of the heat sink 404 are most clearly depicted, as both the top (mounting) surface of the heat sink 404 and the ultrasound-on-chip device 112 are covered by the acoustic lens 104 for illustration purposes. Subsequent views depict these obscured components in further detail, however. For example, FIG. 5 depicts the ultrasonic transducer assembly 110 of FIG. 4, with the acoustic lens 104 removed to reveal the ultrasound-on-chip device 112. The interposer circuit board 402 serves as an electrical interface between the ultrasound-on-chip device 112 and the first and second circuit boards 114, 116 shown in FIG. 2. Connections between a mounting surface of the ultrasound-on-chip device 112 and a first side of the interposer circuit board 402 may be made, for example, using individual wire bonds (not shown) that may in turn be encapsulated by an encapsulant material (not shown). In addition, the interposer circuit board 402 may include one or more connectors 408 configured to mate with corresponding connectors of the first and second circuit boards 114, 116.

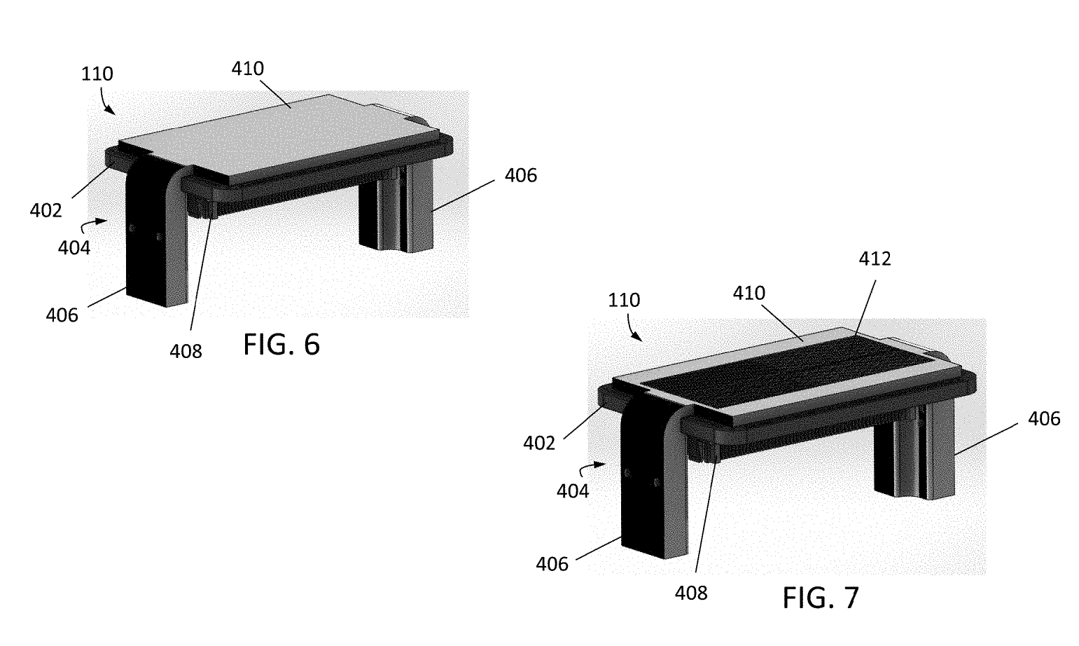

FIG. 6 illustrates the ultrasonic transducer assembly 110 with the ultrasound-on-chip device 112 removed to reveal a planar mounting surface 410 of a heat sink 404. Although the heat sink 404 shown in FIG. 6 provides desired thermal dissipation heat generated by the ultrasound-on-chip device 112, the properties of the heat sink metal affect how much acoustic energy is reflected and how much is absorbed. In this case, the planar (flat) mounting surface 410 may act as an acoustic reflector that redirects unabsorbed acoustic waves back toward the transducers of the ultrasound-on-chip device 112. This is undesirable, since such reflected acoustic waves can contribute to false image data.

Accordingly, FIG. 7 illustrates a heat sink design in accordance with an exemplary embodiment, in which a substantial portion of the mounting surface 410 of the heat sink 404 is a non-planar surface 412. Where geometric features of the mounting surface 410 of the heat sink 404 are made non-planar (as opposed to planar), acoustic sound waves are incident at a non-normal angle with respect to the heat sink surface. Those waves that are not absorbed by the heat sink 406 may be reflected in a direction other than toward the originating transducer and may, in some instances, cancel with one another or at least be scattered in a direction where they may have relatively longer travel times. This in turn may allow for more absorption by the heat sink 406, reducing interference with acoustic waves being detected from the imaged patient. The non-planar surface 412 may, in one embodiment, encompass an area of the mounting surface 410 corresponding to locations of the ultrasonic transducers of the ultrasound-on-chip device 112 when attached to the heat sink 404.

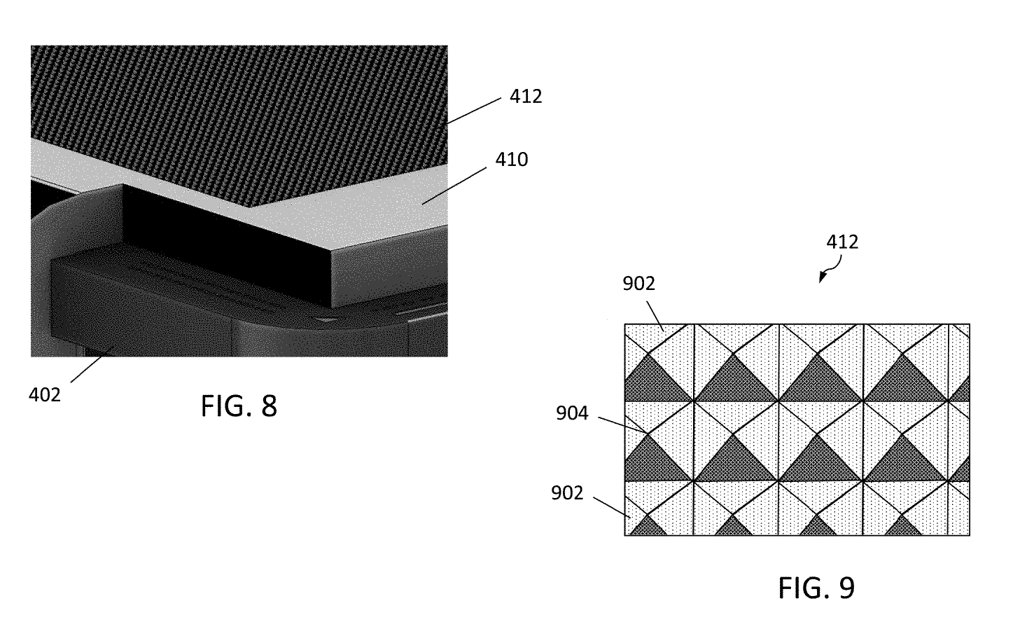

FIG. 8 is an enlarged view of a portion of the heat sink 404 in FIG. 7. The non-planar surface 412 may be defined by forming patterned features in the mounting surface 410. Exemplary techniques for forming the patterned features of the non-planar surface 412 techniques include, but are not limited to, stamping, molding, etching or other microfabrication techniques. The non-planar pattern may include regular features, such as illustrated in FIG. 9, or irregular features as described in further detail hereinafter. The exemplary pattern for the non-planar surface 412 in FIG. 9 includes a plurality of pyramid structures, each having triangular surfaces 902 converging to a single point 904. Other patterns are also contemplated, however.

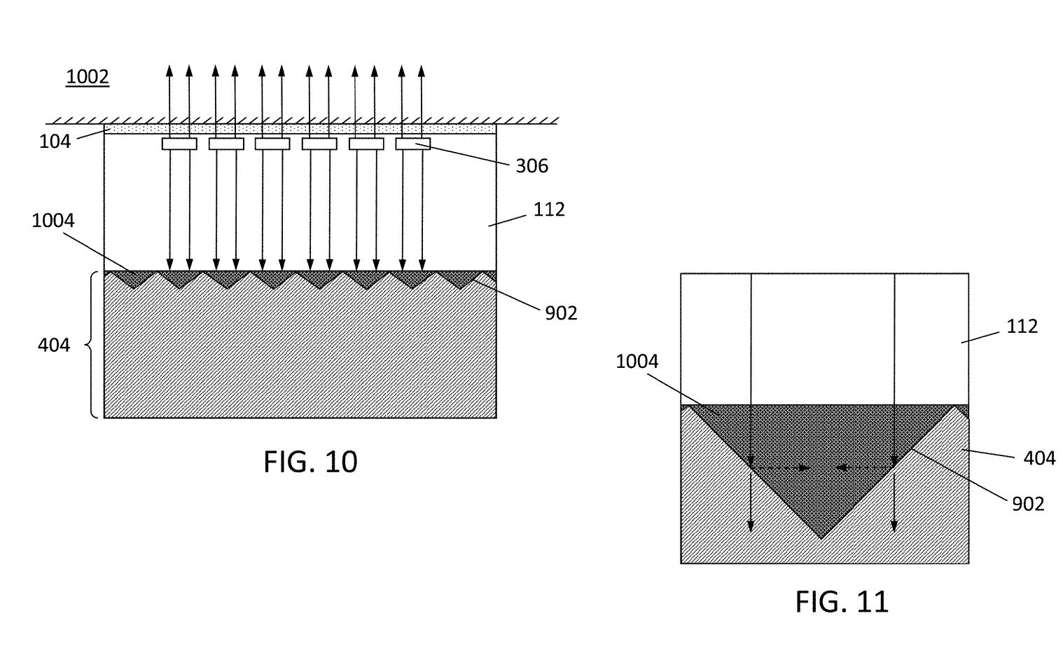

FIG. 10 schematically illustrates the propagation of acoustic waves from an ultrasound-on-chip device 112 attached to the heat sink 404. It should be noted that the device 112 shown in FIG. 10 is a simplified schematic for illustrative purposes, and does differentiate between the transducer portion and the CMOS integrated circuit portion of the device 112, other than depicting the CMUT cavities 306. As will be noted, acoustic waves are transmitted from the transducers in a forward direction into the tissue 1002 of a patient via the acoustic lens 104, as well as in a backward direction through the substrate material of ultrasound-on-chip device 112 toward the interface with the heat sink 404.

An adhesive material 1004 may be used to securely attach the ultrasound-on-chip device 112 to the interface with the heat sink 404. The adhesive material 1004 may be any suitable material known in the art, such as an epoxy material, and optionally a tungsten filled epoxy material or epoxy mixture (with tungsten and/or additional elements) selected for acoustic dampening capabilities. In the enlarged view of FIG. 11, some of the incident acoustic energy incident upon the surfaces 902 may be transmitted through the interface and into the heat sink 404, and some of the incident acoustic energy incident upon the surfaces 902 may be reflected (scattered) in directions away from the transducers. In some cases, reflected acoustic waves may cancel with other reflected acoustic waves.

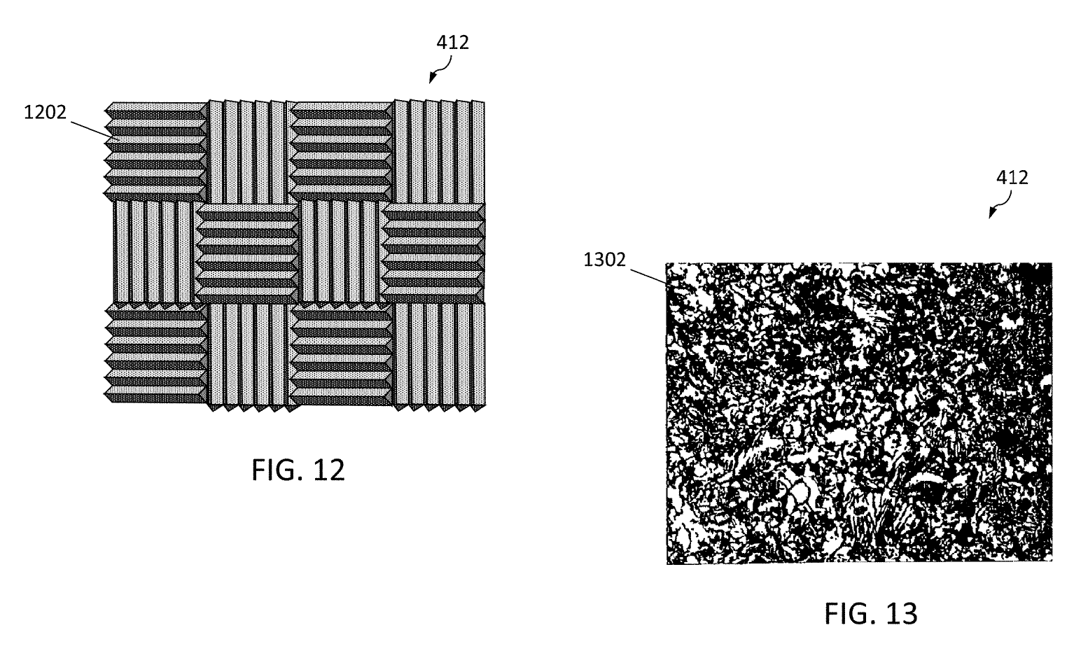

As indicated above, other patterns are also possible for a non-planar surface 412 of the heat sink 404. For example, FIG. 12 illustrates an alternative embodiment of the pattern of the non-planar mounting surface 412. Similar to the embodiment of FIG. 9, a pattern of prism structures 1202 (also referred to as wedge structures) may be formed (e.g., by stamping) on the mounting surface of the heat sink. The structures 1202 may, for example, be formed in groups of repeating arrangements, where structures in adjacent groups have longitudinal apexes disposed orthogonal to one another. Still another embodiment for a non-planar heat sink surface 412 is illustrated in FIG. 13. In this embodiment, the non-planar surface 412 is a sintered surface 1302, which may be formed using a metallic powder to create an irregular surface. Optionally, a tungsten filled epoxy material may also be applied in bonding an ultrasound-on-chip device 112 to the non-planar surfaces 412 of either FIG. 12 or FIG. 13.

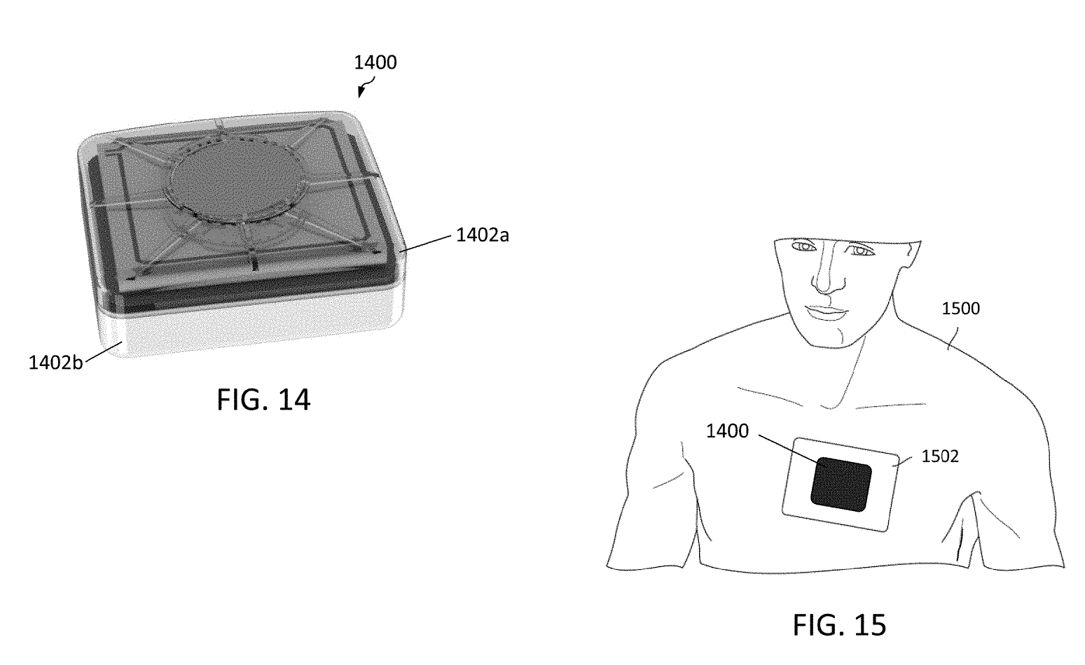

In addition to handheld probe embodiments such as depicted in FIG. 1 and FIG. 2, it is further contemplated that other ultrasound probe form factors may incorporate the above described acoustically diffusing heat sink embodiments. For example, FIG. 14 is a perspective view illustrating an ultrasound probe 1400 that is embodied in a patch configuration, and having an upper housing 1402a and a lower housing 1402b. The probe 1400 is shown coupled to a patient 1500 in FIG. 15, and may be configured to transmit, wirelessly for example, collected ultrasound data to one or more external devices (not shown) for further processing. In the example depicted, the probe 1400 may also be provided with dressing 1502 that provides an adhesive surface for both the probe housing as well as to the skin of the patient. One non-limiting example of such a dressing 1502 is Tegaderm™, a transparent medical dressing available from 3M Corporation. Although not specifically shown in FIG. 15, the lower housing 1402b may include an opening that aligns with a corresponding opening in the dressing 1502 so that transducer elements of the ultrasound probe 1400 may be acoustically coupled to the patient 1500.

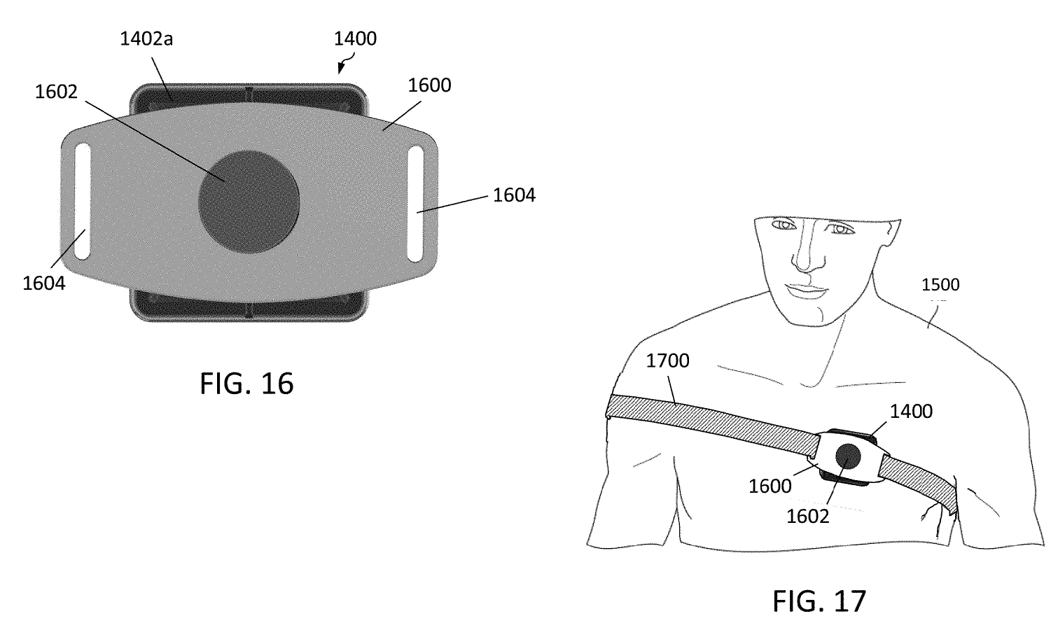

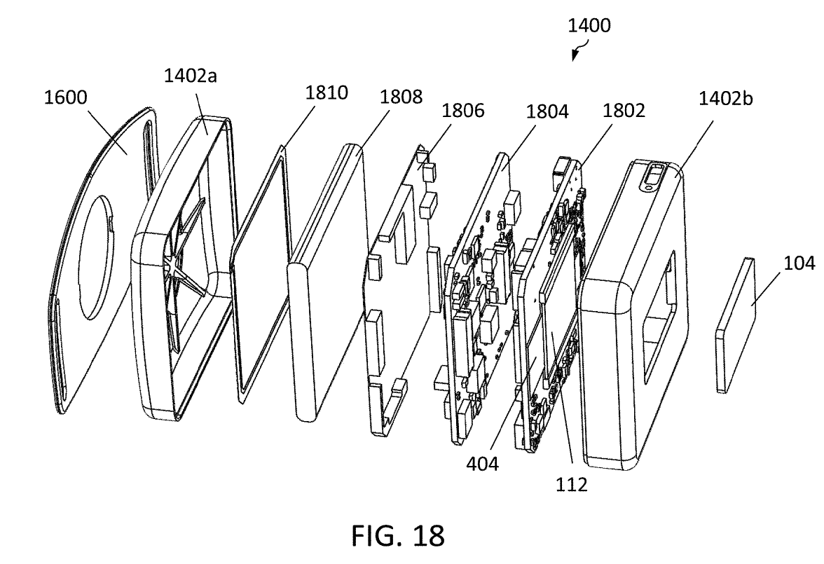

Referring to FIG. 16, an alternative fastening mechanism for the ultrasound probe 1400 is illustrated. In the embodiment shown, the ultrasound probe 1400 further includes a buckle 1600 affixed to the upper housing 1402a via a post 1602 using, for example, a threaded engagement between the buckle 1600 and the post 1602. Other attachment configurations are also contemplated, however. As further shown in FIG. 16, the buckle 1600 includes a pair of slots 1604 that in turn accommodate a strap 1700 (FIG. 17). In this example, the strap 1700 is wrapped around the patient 1500 and appropriately tightened in order to secure the ultrasound probe 1400 to a desired location on the patient 1500 for acquisition of desired ultrasound data.

FIG. 18 illustrates an exploded perspective view of the ultrasound probe 1400 of FIG. 16. For ease of illustration and comparison, similar components with respect to the embodiment of FIG. 1 and FIG. 2 are designated with similar reference numerals. For example, in addition to the upper housing 1402a, lower housing 1402b and buckle, the ultrasound probe 1400 further includes an acoustic lens 104 to cover the ultrasound-on-chip device 112, which in turn is attached to a heat sink device 404. Although not specifically shown in FIG. 18, the mounting surface of the heat sink device 404 may be provided with any of the acoustically diffusing features discussed above, such as for example in any of the embodiments of FIGS. 7, 12 and 13.

In contrast to the handheld probe embodiment of FIGS. 1-8 in which the ultrasonic transducer assembly 110 includes an interposer circuit board 402, the ultrasound-on-chip device 112 and heat sink device 404 are attached directly to a first circuit board 1802. In addition, the ultrasound probe 1400 further includes, by way of example, a second circuit board 1804 (e.g., for power supply components), an insulator board 1806, battery 1808 and antenna 1810 (e.g., to enable wireless communication to and from the ultrasound probe 1400). In any case, it will be appreciated that the above described thermal and acoustic benefits provided by the heat sink device 404 are applicable to ultrasound probes of various form factors.

The techniques described herein are exemplary, and should not be construed as implying any particular limitation on the present disclosure. It should be understood that various alternatives, combinations and modifications could be devised by those skilled in the art from the present disclosure. For example, steps associated with the processes described herein can be performed in any order, unless otherwise specified or dictated by the steps themselves. The present disclosure is intended to embrace all such alternatives, modifications and variances that fall within the scope of the appended claims.

What is claimed is:

1. An apparatus, comprising:
a heat sink device having a non-planar mounting surface; and
an ultrasonic transducer substrate attached to the non-planar mounting surface of the heat sink device;
wherein the non-planar mounting surface of the heat sink device is configured to diffuse acoustic waves that are incident thereupon.

2. The apparatus of claim 1, wherein the ultrasonic transducer substrate comprises a portion of an ultrasound-on-chip device attached to the non-planar mounting surface of the heat sink device, the ultrasound-on-chip device further comprising the ultrasonic transducer substrate bonded to an integrated circuit substrate.

3. The apparatus of claim 1, wherein the non-planar mounting surface of the heat sink device comprises a pattern.

4. The apparatus of claim 3, wherein the pattern comprises a plurality of pyramid structures.

5. The apparatus of claim 3, wherein the pattern comprises a plurality of prism structures.

6. The apparatus of claim 1, wherein the non-planar mounting surface of the heat sink device comprises a plurality of irregular features.

7. The apparatus of claim 6, wherein plurality of irregular features comprises a sintered surface.

8. The apparatus of claim 1, further comprising an adhesive material that attaches the ultrasound-on-chip device to the non-planar mounting surface of the heat sink device.

9. The apparatus of claim 8, wherein the adhesive material comprises an epoxy material.

10. The apparatus of claim 8, wherein the adhesive material comprises a tungsten filled epoxy material.

11. The apparatus of claim 1, wherein the ultrasound transducer substrate is further configured to accommodate a solid state monolithic ultrasound transducer.

12. An ultrasound probe, comprising:
a housing; and
an ultrasonic transducer assembly disposed within the housing, the ultrasonic transducer assembly further comprising a metal heat sink device having a non-planar mounting surface, and an ultrasonic transducer substrate attached to the non-planar mounting surface of the heat sink device;
wherein the non-planar mounting surface of the heat sink device is configured to diffuse acoustic waves that are incident thereupon.

13. The ultrasound probe of claim 12, wherein the ultrasonic transducer substrate comprises a portion of an ultrasound-on-chip device attached to the non-planar mounting surface of the heat sink device, the ultrasound-on-chip device further comprising the ultrasonic transducer substrate bonded to an integrated circuit substrate.

14. The ultrasound probe of claim 13, wherein the non-planar mounting surface of the heat sink device comprises a stamped pattern.

15. The ultrasound probe of claim 14, wherein the stamped pattern comprises a plurality of pyramid structures.

16. The ultrasound probe of claim 14, wherein the stamped pattern comprises a plurality of prism structures.

17. The ultrasound probe of claim 12, wherein the non-planar mounting surface of the heat sink device comprises a plurality of irregular features.

18. The ultrasound probe of claim 17, wherein the plurality of irregular features comprises a sintered surface.

19. The ultrasound probe of claim 12, further comprising an adhesive material that attaches the ultrasound-on-chip device to the non-planar mounting surface of the heat sink device.

20. The ultrasound probe of claim 19, wherein the adhesive material comprises an epoxy material.

21. The ultrasound probe of claim 19, wherein the adhesive material comprises a tungsten filled epoxy material.

22. The ultrasound probe of claim 12, wherein the housing comprises a handheld probe.

23. The ultrasound probe of claim 12, wherein the housing comprises a patch configured to be affixed to a patient.

24. The ultrasound probe of claim 12, wherein the ultrasound transducer assembly further comprises a solid state monolithic ultrasound transducer.

25. The apparatus of claim 24, wherein the solid state monolithic ultrasound transducer further comprises a plurality of capacitive ultrasound transducers bonded with an integrated circuit.
